(19) United States
(12) Patent Application Publication
(10) Pub. No.: US 2018/0127638 A1
(43) Pub. Date: May 10, 2018

(54) TIME RELEASED DELIVERY OF FUNCTIONAL CHEMICALS

(71) Applicant: Covestro LLC, Pittsburgh, PA (US)

(72) Inventors: James A. Thompson-Colón, Moon Township, PA (US); Michael Jeffries, Follansbee, WV (US); Mark D. Conner, Kempton, PA (US); David P. Zielinski, Cranberry Township, PA (US)

(73) Assignee: Covestro LLC

(21) Appl. No.: 15/798,851

(22) Filed: Oct. 31, 2017

Related U.S. Application Data
(60) Provisional application No. 62/418,427, filed on Nov. 7, 2016.

(51) Int. Cl. C09K 8/62; C09D 7/12; C09K 8/035; C09K 8/60; C09K 8/42; C09J 11/06; C09J 11/08; C07D 223/10; C07D 249/08; C07C 251/38; C07D 231/12; C07C 237/06; E21B 37/06; E21B 41/02; E21B 43/267

(52) U.S. Cl. CPC C09K 8/62; E21B 33/13; C09D 7/125; C09K 8/035; C09K 8/60; C09K 8/42; C09J 11/06; C09J 11/08; C07D 223/10; C07D 249/08; C07C 251/38; C07D 231/12; C07C 237/06; E21B 37/06; E21B 41/02; E21B 43/267; C09D 7/1233

(57) ABSTRACT

The present invention provides a composition comprising an isocyanate, and a functional chemical, wherein the functional chemical is attached to the isocyanate at an active hydrogen atom of the functional chemical. The present invention offers a way of assuring that the isocyanate-attached functional chemical is released slowly over time at any stage of the oil and gas well lifecycle.

### CROSS REFERENCE TO RELATED APPLICATION(S)

This Application claims the benefit of U.S. Provisional Application Ser. No. 62/418,427, filed Nov. 7, 2016, the entire contents of which are incorporated herein by reference.

### FIELD OF THE INVENTION

The present invention relates in general to oil and gas production and more specifically to a functional chemical having an active hydrogen which is attached at its active hydrogen to an isocyanate.

### BACKGROUND OF THE INVENTION

A typical oil and gas well has five stages during its life cycle: drilling; wellbore completion; cementing; stimulation; and production.

Drilling is the process of creating a hole, which can range from as little as five inches to as much as three feet in diameter, with a drilling rig. The drilling rig rotates a drill bit which hangs from a drill string. A drill string is a column, or string, of drill pipe that transmits drilling fluid (via mud pumps) and torque (via a top drive) to the drill bit.

Wellbore completion is the process of cleaning out the wellbore upon completion of the drilling operations to minimize reservoir damage, maximize drilling fluid recovery and make the well ready for production. This stage of the life cycle typically involves preparing the bottom of the hole to the required specifications, running in production tubing and associated downhole tools and perforating and stimulating as required.

Cementing is the process of mixing and pumping a cement slurry downhole to secure the casing and ensuring zone isolation throughout the length of the wellbore. Cementing protects and seals the wellbore and is most commonly used to permanently shut off water penetration into the well.

Stimulation is the process of well intervention that is performed to improve production rates by increasing the flow of hydrocarbons into the wellbore. Stimulations may occur multiple times during the lifetime of a given well. Well stimulation is typically done through one of three methods: hydraulic fracturing; acid fracturing and matrix acidizing.

Hydraulic fracturing is one of the most complex oilfield services employed today, requiring equipment to transport and store water and chemicals, prepare the fracturing fluid, blend the fluid with proppant, pump the fluid down the well and monitor the treatment.

Typically, functional chemicals are added to the fracing fluid or coated onto proppants to provide desired attributes to the fluid or proppant. Such chemicals include, corrosion inhibitors, defoamers, demulsifiers, emulsifiers, dispersants, filtrate reducers, water clarifiers/deoilers, fluid-loss additives, flocculants, foaming agents, defoaming agents, scale inhibitors, shale control inhibitors, viscosifiers, thinners, wax inhibitors, asphaltene inhibitors, hydrate inhibitors, biocides, rheology modifiers, trace/tagging agents, co-surfactants, surfactants, co-solvents, H₂S scavengers, ion exchange resins, cement gas blocking agents, cement retarders, de-watering additives, bentonite extenders, friction reducers, gelling agents, gel breakers, iron control additives, clay stabilizers, and iron sulfide dissolvers.

Production is the most important stage of the well's life, wherein oil and gas are produced. At this stage, the oil rigs and workover rigs which were used in drilling and completing the well will have been moved off the wellbore, and the top will be outfitted with a collection of valves known as a “Christmas tree” or production tree to regulate pressures, control flows, and allow access to the wellbore if further completion work is needed. The hydrocarbon flow can be connected by the outlet valve of the production tree to a distribution network of pipelines and tanks to supply the product to refineries, natural gas compressor stations, or oil export terminals.

It would be desirable to have a way to release functional chemicals slowly over time in any or all of these stages of the oil and gas well life cycle.

### SUMMARY OF THE INVENTION

Accordingly, the present invention provides a functional chemical having an active hydrogen which is attached at its active hydrogen to an isocyanate. The present invention offers a variety of ways of assuring that the isocyanate-attached functional chemical is released into the oil and gas well at any stage of its lifecycle and released slowly over time. Such functional chemicals include, but are not limited to, demulsifiers, emulsifiers, corrosion inhibitors, dispersants, filtrate reducers, water clarifiers/deoilers, fluid-loss additives, flocculants, foaming agents, defoaming agents, scale inhibitors, shale control inhibitors, viscosifiers, thinners, wax inhibitors, asphaltene inhibitors, hydrate inhibitors, biocides, rheology modifiers, trace/tagging agents, co-surfactants, co-solvents, H₂S scavengers, ion exchange resins, cement gas blocking agents, cement retarders, de-watering additives, bentonite extenders, friction reducers, gelling agents, gel breakers, iron control additives, clay stabilizers, and iron sulfide dissolvers.

These and other advantages and benefits of the present invention will be apparent from the Detailed Description of the Invention herein below.

### DETAILED DESCRIPTION OF THE INVENTION

The present invention will now be described for purposes of illustration and not limitation. Except in the operating examples, or where otherwise indicated, all numbers expressing quantities, percentages, and so forth in the specification are to be understood as being modified in all instances by the term “about.”

Any numerical range recited in this specification is intended to include all sub-ranges of the same numerical precision subsumed within the recited range. For example, a range of “1.0 to 10.0” is intended to include all sub-ranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited in this specification is intended to include all lower numerical limitations subsumed therein and any minimum numerical limitation recited in this specification is intended to include all higher numerical limitations subsumed therein. Accordingly, Applicants reserve the right to amend this specification, including the claims, to expressly recite any sub-range subsumed within the ranges expressly recited herein. All such ranges are intended to be inherently described in this specification such that amending to expressly recite any such sub-ranges would comply with the requirements of 35 U.S.C. § 112(a), and 35 U.S.C. § 132(a).

Any patent, publication, or other disclosure material identified herein is incorporated by reference into this specification in its entirety unless otherwise indicated, but only to the extent that the incorporated material does not conflict with existing definitions, statements, or other disclosure material expressly set forth in this specification. As such, and to the extent necessary, the express disclosure as set forth in this specification supersedes any conflicting material incorporated by reference herein. Any material, or portion thereof, that is said to be incorporated by reference into this specification, but which conflicts with existing definitions, statements, or other disclosure material set forth herein, is only incorporated to the extent that no conflict arises between that incorporated material and the existing disclosure material. Applicants reserve the right to amend this specification to expressly recite any subject matter, or portion thereof, incorporated by reference herein.

Reference throughout this specification to “various non-limiting embodiments,” “certain embodiments,” or the like, means that a particular feature or characteristic may be included in an embodiment. Thus, use of the phrase “in various non-limiting embodiments,” “in certain embodiments,” or the like, in this specification does not necessarily refer to a common embodiment, and may refer to different embodiments. Further, the particular features or characteristics may be combined in any suitable manner in one or more embodiments. Thus, the particular features or characteristics illustrated or described in connection with various or certain embodiments may be combined, in whole or in part, with the features or characteristics of one or more other embodiments without limitation. Such modifications and variations are intended to be included within the scope of the present specification.

The grammatical articles “a”, “an”, and “the”, as used herein, are intended to include “at least one” or “one or more”, unless otherwise indicated, even if “at least one” or “one or more” is expressly used in certain instances. Thus, these articles are used in this specification to refer to one or more than one (i.e., to “at least one”) of the grammatical objects of the article. By way of example, and without limitation, “a component” means one or more components, and thus, possibly, more than one component is contemplated and may be employed or used in an implementation of the described embodiments. Further, the use of a singular noun includes the plural, and the use of a plural noun includes the singular, unless the context of the usage requires otherwise.

The present invention is directed generally to a functional chemical having an active hydrogen which is attached at its active hydrogen to an isocyanate and to methods of producing those isocyanate-attached functional chemicals. Such isocyanate-attached functional chemicals in various embodiments may be added to a coating formulation which is applied to proppants. The present invention is also directed to methods for preparing such coated proppants, and methods for using those proppants to ameliorate various conditions in the downhole environment, for example, corrosion, well-souring, etc. The functional chemicals may in some embodiments be added to a resin-containing dispersion and a coating made with that dispersion which is applied to the proppants to impart various desired properties to the proppants.

In other embodiments, the isocyanate-attached functional chemicals may be added to fracing fluid and introduced into the downhole environment in that manner Further, the isocyanate-attached functional chemicals may simply be introduced into the well in a dry form and allowed to mix with the fluid already within the well, e.g. fracing fluid, water, a hydrocarbon, etc.

In certain embodiments, the isocyanate-attached functional chemicals are introduced into one or more of the stages of the oil and gas well lifecycle: drilling; wellbore completion; cementing; stimulation; and production by mixing with the liquid used in that stage or may be added in a dry form and allowed to mix with the residual liquid during the stage(s).

To define more clearly the terms and concepts disclosed herein, the following definitions are provided. To the extent that any definition or usage provided by any document incorporated herein by reference conflicts with the definition or usage provided herein, the definition or usage provided herein controls.

For those embodiments of the present invention where the isocyanate-attached functional chemicals are in a coating applied to a proppant, the terms “particle”, “particulate”, “particulate material” and the like, when unmodified, are used herein to indicate the base material which, when having a coating, forms a “proppant.” For example, hydraulic fracturing (fracing) sand is a material that is often referred to in the art as a “proppant”, but in this disclosure, it is referred to as a “particle.” The terms “proppant”, “proppant particle”, “coated proppant”, “adhered proppant”, and the like, are reserved for coated particles in accordance with the teachings of this invention.

The term “free-flowing” is used herein to mean that the proppant particles do not agglomerate appreciably, and generally remain as discrete, individual proppant particles. Proppants of the present invention are “free-flowing” at ambient conditions, i.e., at a temperature of about 20-25° C. and at atmospheric pressure. The flowability of the solid particles can be measured using a test method such as the American Foundrymen's Society Procedure 227-87-S, entitled “Moldability of Molding Sand Mixtures” as found in the Mold & Core Test Handbook, 2nd edition (1989), which is incorporated herein by reference. Briefly, the test procedure involves placing a 200 g sample of solid particles in a cylindrical 8-mesh screen of a rotary screen device driven by a 57 rpm motor. The screen was rotated for 10 sec. In accordance with this test, the moldability index is equal to the weight of the product passing through the screen divided by the original weight charged to the screen chamber (i.e., 200 g). If all of the material passes through the screen, the moldability index is 100%. In accordance with the present invention, free-flowing proppants have a moldability index of greater than about 80% at ambient conditions. For instance, the proppants disclosed herein can have a moldability index greater than about 85%, or greater than about 90%. In some aspects of this invention, the coated proppants have a moldability index of greater than about 95%, or alternatively, greater than about 98%. Generally, solid materials that are not free-flowing have a moldability index of less than about 50%. Such materials can, in some cases, have a moldability index of less than about 40%, or less than about 25%.

Although any methods, devices, and materials similar or equivalent to those described herein can be used in the practice or testing of the invention, the typical methods, devices and materials are herein described.

Although compositions and methods are described in terms of “comprising” various components or steps, the compositions and methods can also “consist essentially of” or “consist of” the various components or steps.

In certain embodiments, the present invention is directed to dispersion-coated proppants, methods for preparing the coated proppants, and methods for using those proppant particles to alter various conditions within the well-bore. A coated proppant in accordance with one aspect of the present invention comprises (i) a particle, and (ii) a resin-containing dispersion adhered to the particle. The resin containing dispersion will also contain the isocyanate-attached functional chemical.

The coated particles of certain embodiments of the present invention can be prepared by any of a variety of processes, including batch, semi-continuous, or continuous processes. Batch, continuous mixers or in-line where the sand is effectively agitated sufficient to spread the dispersion onto the particle surface may be used to prepare the coated particles of some embodiments of the invention. Suitable methods of preparing the particles include, but are not limited to, spraying, slurrying, flooding, and simply adding solution to bulk proppant and stirring. Application temperatures may be from about 4.4° C. (40° F.)—the coating solution must be flowable but protected from freezing-up to as high as approximately 232.22° C. (450° F.). At temperatures above 100° C. (212° F.), i.e., the boiling point of water, the required contact time of the solution should be limited due to the rapid evolution of water from the mixture. At lower application temperatures, the dispersion-coated particle mixture requires longer drying times or the addition of heat to speed the drying process.

Various downhole environment embodiments of the invention utilize a masterbatch in which a portion of the particles are coated with an isocyanate-attached functional chemical containing dispersion at a higher concentration of functional chemical than is desired in the final particle slurry. These coated particles are then mixed with uncoated particles and the mixture used in the desired application. In this manner, for example, a smaller volume masterbatch of corrosion inhibiting isocyanate-attached functional chemical containing dispersion coated sand may be transported to an oil or gas well site where it may be mixed with a larger volume of uncoated sand and introduced into a well bore. The mixing may optionally take place in stages so as to vary the concentration of corrosion inhibiting isocyanate-attached functional chemical at different levels of the well bore as the sand is introduced.

The present invention is not limited to any specific type of particulate material for use as the proppant substrate, so long as the particle has sufficient strength to withstand the stresses, such as elevated temperature and pressure, often encountered in oil and gas recovery applications.

In one aspect of the present invention, the particle of the coated proppant is a sand, a naturally occurring mineral fiber, a ceramic, a bauxite, a glass, a metal bead, a walnut hull, a composite particle, and the like. For instance, the sand can be graded sand or a resin-coated sand. These dispersion coated sands can serve as particles in the present invention. A ceramic can include both porous and non-porous ceramic materials, and a bauxite can include sintered bauxite materials.

Composite particles are an agglomeration of smaller, fine particles held together by a binder, and such composite particles can be the particulate material in the present invention. Compositions containing coated proppants can employ mixtures or combinations of more than one type of particle, for instance, both a sand and a ceramic can be coated and then mixed to form a composition of proppants. It is contemplated that any particulate material suitable for use in proppant applications can be used in the present invention, regardless of the specific gravity of the particle, although it can be beneficial in certain applications to have a lower specific gravity to increase the distance that the proppants can be carried into a formation prior to settling.

In another aspect, the particle is either a porous ceramic or porous polymer particle. Such particles are described in, for example, U.S. Pat. Nos. 7,426,961 and 7,713,918, the disclosures of which are incorporated herein by reference in their entirety. These porous ceramic or porous polymer materials can be of natural origin or can be produced synthetically. Although the use of such materials is not limited by specific gravity, the specific gravity of these materials is generally less than about 3 g/cc, or less than about 2.7 g/cc. In another aspect, the specific gravity of the porous particle is less than about 2.5 g/cc, for example, less than about 2.2 g/cc.

The particle size of the particle used to produce the coated proppant of the present invention generally falls within a range from about 100 μm to about 3000 μm (about 3 mm). In another aspect, the particle size is from about 125 μm to about 2500 μm, from about 150 μm to about 2000 μm, or from about 175 μm to about 1500 μm. In yet another aspect, the particle of the coated proppant of the present invention has a particle size that falls within a narrower range of about 200 μm to about 1000 μm, for example, about 250 μm to about 800 μm, or from about 300 μm to about 700 μm.

In another aspect of this invention, the particles generally have a mesh size from about 8 and about 100, based on the U.S. Standard Sieve Series. For example, in a distribution of such particles which can be added to a treating fluid for use in a subterranean formation, at least about 90% by weight of the particles have a particle size falling within the range from about 8 to about 100 mesh. In accordance with another aspect of the present invention, at least about 95% by weight of the particles in a proppant composition have a size within the range from about 8 to about 100 mesh. Further, 90% by weight or more (e.g., 95% or more) of the particles in a proppant composition can have a size within the 20 to 40 mesh range in another aspect of this invention.

In a different aspect, the particle in the coated proppant has a size in the range from about 8 to about 140 mesh, from 10 to about 120 mesh, from about 10 to about 100 mesh, or from about 14 to about 80 mesh. In other aspects of this invention, the particle is in a range from about 18 to about 60 mesh, or from about 20 mesh to about 40 mesh. In another aspect, there is less than about 10% by weight, for example, 5% by weight of less, of particles in a coated proppant composition having a size of less than about 20 mesh or greater than about 50 mesh. The proppants of the present invention generally comprise particles which are not limited to any particular material or size.

The coated particles described herein can be used in a variety of applications including, for example, use as a component of a coating, adhesive, or sealant composition, in which the coated particles are dispersed in a binder resin, such as any binder resin known to those skilled in the art of such compositions.

In certain embodiments, however, the coated particles of the present invention are thought to be particularly suitable for use in hydraulic fracturing of a geologic formation. In these embodiments, the coated particles may be combined with a carrier fluid, such as water and/or a hydrocarbon, and the mixture injected at elevated pressure into a well bore to an underground geologic formation. When the pressure in the formation resulting from the injection exceeds the strength of the formation, a fracture is formed and the coated particles, i.e., proppant, are placed in the formation in an effort to maintain the fracture in a propped open position when the injection pressure is released. Upon ceasing the injection of fluid, it is desired that the proppant forms a pack that serves to hold open the fractures and providing a highly conductive channel through which a desired material, such as water, oil, or gas (including natural gas) can flow to the well bore for retrieval.

In various embodiments, therefore, the coated particles are used in a method of forming a proppant composition that includes suspending the particles described herein in a carrier fluid to form a suspension and injecting the suspension into an underground geologic formation. The suspended particles will provide a slow release of functional chemical(s).

The coated particles described herein can be injected as the sole proppant or as a partial replacement for an existing proppant. For example, if desired, the coated particles described herein may comprises 1 to 99 percent by weight, such as 10 to 90 percent by weight, or, in some cases, 10 to 50 percent by weight, based on the total weight of the proppant present in the composition that is injected into the well bore. In some embodiments, an uncoated proppant is first placed in a well, and thereafter a proppant of the coated particles described herein is placed in the fracture nearest to the wellbore or fracture openings.

The coated particles of the present invention are presently thought to provide several advantages, particularly in the context of hydraulic fracturing. Depending on the functional chemical coated onto the proppant, the proppant may have anti-microbial properties, dispersant properties, corrosion inhibiting properties, hydrogen sulfide scavenging properties, gelling properties, and/or scale inhibiting properties, among others.

As used herein, the term “coating” refers to a set of chemical components that may be mixed to form an active coating composition that may be applied and cured to form a coating. As used herein, the term “coating composition” refers to a mixture of chemical components that will dry by eliminating water and/or co-solvent. Accordingly, a coating composition may be formed from a coating system by mixing the chemical components comprising the coating system. Furthermore, when a list of constituents is provided herein that are individually suitable for forming the components of the coating system or coating composition discussed herein, it should be understood that various combinations of two or more of those constituents, combined in a manner that would be known to those of ordinary skill in the art reading the present specification, may be employed and is contemplated.

As used herein, the term “polyurethane” refers to polymeric or oligomeric materials comprising urethane groups, urea groups, or both. Accordingly, as used herein, the term “polyurethane” is synonymous with the terms polyurea, poly(urethane/urea), and modifications thereof. The term “polyurethane” also refers to crosslinked polymer networks in which the crosslinks comprise urethane and/or urea linkages, and/or the constituent polymer chains comprise urethane and/or urea linkages. Carbodiimide crosslinking as is known to those skilled in the art is also contemplated in the dispersion coated proppants of the invention.

As used herein, the term “dispersion” refers to a composition comprising a discontinuous phase distributed throughout a continuous phase. For example, “waterborne dispersion” and “aqueous dispersion” refer to compositions comprising particles or solutes distributed throughout liquid water. Waterborne dispersions and aqueous dispersions may also include one or more co-solvents in addition to the particles or solutes and water. As used herein, the term “dispersion” includes, for example, colloids, emulsions, suspensions, sols, solutions (i.e., molecular or ionic dispersions), and the like. Resin-containing dispersion in the present invention may be applied at 0.05 wt. % to 10.0 wt. % resin solids based on the weight of the proppant.

In certain embodiments, the thickness of the resin-containing dispersion coating on the particle (substrate) is 200 nm to 450 nm. In other embodiments, the thickness is 221 nm to 441 nm.

As used herein, the term “polyisocyanate” refers to compounds comprising at least two free isocyanate groups. Polyisocyanates include diisocyanates and diisocyanate reaction products comprising, for example, biuret, isocyanurate, uretdione, urethane, urea, iminooxadiazine dione, oxadiazine trione, carbodiimide, acyl urea, and/or allophanate groups.

As used herein, the term “polyol” refers to compounds comprising at least two free hydroxy groups. Polyols include polymers comprising pendant and/or terminal hydroxy groups. As used herein, the term “polyamine” refers to compounds comprising at least two free amine groups. Polyamines include polymers comprising pendant and/or terminal amine groups.

Water-dispersible polyisocyanates include polyisocyanates that may form an aqueous dispersion with the aid of organic co-solvents, protective colloids, and/or external emulsifiers under high shear conditions. Water-dispersible polyisocyanates also include polyisocyanates that are hydrophilically-modified with covalently linked internal emulsifiers.

The polyisocyanate useful in the present invention may comprise any organic polyisocyanates having aliphatically, cycloaliphatically, araliphatically, and/or aromatically bound free isocyanate groups, which are liquid at room temperature or are dispersed in a solvent or solvent mixture at room temperature. In various embodiments, the polyisocyanate may have a viscosity of from 10-15000 mPa·s at 23° C., 10-5000 mPa·s at 23° C., or 50-1000 mPa·s at 23° C. In some embodiments, the polyisocyanate may comprise polyisocyanates or polyisocyanate mixtures having exclusively aliphatically and/or cycloaliphatically bound isocyanate groups with an (average) NCO functionality of 2.0-5.0 and a viscosity of from 10-5000 mPa·s at 23° C., 50-1000 mPa·s at 23° C., or 100-1000 mPa·s at 23° C.

In certain embodiments, the polyisocyanate may comprise polyisocyanates or polyisocyanate mixtures based on one or more aliphatic or cycloaliphatic diisocyanates, such as, for example, ethylene diisocyanate; 1,4-tetramethylene diisocyanate; 1,6-hexamethylene diisocyanate (HDI); 2,2,4-trimethyl-1,6-hexamethylene diisocyanate; 1,12-dodecamethylene diisocyanate; 1-isocyanato-3-isocyanatomethyl-3,5,5-trimethyl-cyclohexane (isophorone diisocyanate or IPDI); bis-(4-isocyanatocyclohexyl)methane (Hi2MDI); cyclohexane 1,4-diisocyanate; bis-(4-isocyanato-3-methyl-cyclohexyl)methane; PDI (pentane diisocyanate—bio-based) isomers of any thereof; m-xylylene diisocyanate (XDI) or combinations of any thereof. In various embodiments, the polyisocyanate component may comprise polyisocyanates or polyisocyanate mixtures based on one or more aromatic diisocyanates, such as, for example, benzene diisocyanate; toluene diisocyanate (TDI); diphenylmethane diisocyanate (MDI); isomers of any thereof; or combinations of any thereof. In various embodiments, the polyisocyanate component may comprise a triisocyanate, such as, for example, 4-isocyanatomethyl-1,8-octane diisocyanate (triisocyanatononane or TIN); isomers thereof; or derivatives thereof.

Additional polyisocyanates (including various diisocyanates) that may find utility in the polyurethane dispersion useful in the present invention may include the polyisocyanates described in U.S. Pat. Nos. 5,075,370; 5,304,400; 5,252,696; 5,750,613; and 7,205,356, each of which is incorporated by reference herein. Combinations of any of the above-identified and incorporated polyisocyanates may also be used to form a polyurethane dispersion useful herein.

The di- and tri-isocyanates indicated may be used as such, or as derivative polyisocyanates comprising biuret, isocyanurate, uretdione, urethane, urea, iminooxadiazine dione, oxadiazine trione, carbodiimide, acyl urea, and/or allophanate groups. In various embodiments, derivative polyisocyanates comprising biuret, isocyanurate, uretdione, urethane, iminooxadiazine dione, oxadiazine trione, carbodiimide, acyl urea, and/or allophanate groups are included in the polyisocyanate coating. In various embodiments, the polyisocyanate component comprises one or more of the above-identified structural groups prepared from IPDI, HDI, H₁₂MDI, and/or cyclohexane 1,4-diisocyanate.

The polyisocyanate may be hydrophilically-modified to be water-dispersible. Hydrophilically-modified water-dispersible polyisocyanates are obtainable, for example, by covalent modification with an internal emulsifier comprising anionic, cationic, or nonionic groups.

Polyether urethane type water-dispersible polyisocyanates may be formed, for example, from a reaction between polyisocyanates and less than stoichiometric amounts of monohydric polyalkylene oxide polyether alcohols. The preparation of such hydrophilically-modified polyisocyanates is described, for example, in U.S. Pat. No. 5,252,696, which is incorporated by reference herein. Polyether allophanate type water-dispersible polyisocyanates may be formed, for example, from a reaction between a polyalkylene oxide polyether alcohol and two polyisocyanate molecules under allophanation conditions. The preparation of such hydrophilically-modified polyisocyanates is described, for example, in U.S. Pat. No. 6,426,414, which is incorporated by reference herein. The polyalkylene oxide polyether alcohol used to prepare polyether type hydrophilically-modified water-dispersible polyisocyanates may comprise, for example, polyethylene oxide residues and/or polypropylene oxide residues.

Polyisocyanates may also be covalently modified with ionic or potentially ionic internal emulsifying groups to form hydrophilically-modified water-dispersible polyisocyanates. The ionic or potentially ionic groups may be cationic or anionic. As used herein, the term “ionic or potentially ionic group” refers to a chemical group that is nonionic under certain conditions and ionic under certain other conditions. For example, in various embodiments, the ionic group or potentially ionic group may comprise a carboxylic acid group; a carboxylate group; a sulfonic acid group; a sulfonate group; a phosphonic acid group; a phosphonate group; or combinations of any thereof. In this regard, for example, carboxylic acid groups, sulfonic acid groups, and phosphonic acid groups are potentially ionic groups, whereas, carboxylate groups, sulfonate groups, and phosphonate groups are ionic groups in the form of a salt, such as, for example, a sodium salt.

For example, carboxylate (carboxylic acid) groups, sulfonate (sulfonic acid) groups, or phosphonate (phosphonic acid) groups may be covalently introduced into polyisocyanates to form hydrophilically-modified water-dispersible polyisocyanates. The ionic or potentially ionic groups may be introduced through a reaction between the isocyanate groups of the polyisocyanate and less than stoichiometric amounts of amino-functional or hydroxy-functional carboxylic acids, sulfonic acids, phosphonic acids, or salts thereof. Examples include, but are not limited to dimethylolpropionic acid (DMPA), N-(2-aminoethyl)-2-aminoethane sulfonic acid (AAS); N-(2-aminoethyl)-2-aminopropionic acid; 2-(cyclohexyl-amino)-ethane sulfonic acid; 3-(cyclohexyl-amino)-1-propane sulfonic acid (CAPS); 2-aminoethylphosphonic acid; or the salts thereof.

If free carboxylic acids, sulfonic acids, or phosphonic acids are incorporated in the polyisocyanate, then the acids may be neutralized with a neutralizing agent, such as, for example, tertiary amines, including, but not limited to, trialkyl-substituted tertiary amines The preparation of hydrophilically-modified water-dispersible polyisocyanates is described, for example, in U.S. Pat. No. 6,767,958, which is incorporated by reference herein. Water-dispersible polyisocyanate mixtures based on triisocyanatononane (TIN) are described in International Patent Application Publication No. WO01/62819, which is incorporated by reference herein.

The NCO content of nonionic type hydrophilically-modified water-dispersible polyisocyanates may be from 5 to 25 weight percent of the polyisocyanate molecule. The NCO content of ionic type hydrophilically-modified water-dispersible polyisocyanates may be from 4 to 26 weight percent of the polyisocyanate molecule.

The polyisocyanates may also be partially blocked with compounds that are reversibly reactive with isocyanate groups. Suitable blocking agents for polyisocyanates include, for example, monohydric alcohols such as methanol, ethanol, butanol, hexanol, cyclohexanol, benzyl alcohol, oximes such as acetoxime, methyl ethyl ketoxime, cyclohexanone oxime, lactams such as E-caprolactam, phenols, amines such as diisopropylamine or dibutylamine, dimethylpyrazole or triazole, as well as malonic acid dimethyl ester, malonic acid diethyl ester or malonic acid dibutyl ester.

To produce the isocyanate-attached functional chemical of the present invention, a monoisocyanate or polyisocyanate is reversibly blocked with a blocking agent. The blocked monoisocyanate or polyisocyanate is unblocked and a functional chemical having an active hydrogen is reacted at the active hydrogen resulting in the isocyanate-attached functional chemical.

Suitable blocking agents for producing the isocyanate-attached functional chemical of the present invention include, but are not limited to, monohydric alcohols such as methanol, ethanol, butanol, hexanol, cyclohexanol, benzyl alcohol, oximes such as acetoxime, methyl ethyl ketoxime, cyclohexanone oxime, lactams such as ϵ-caprolactam, phenols, amines such as diisopropylamine or dibutylamine, dimethylpyrazole or triazole, as well as malonic acid dimethyl ester, malonic acid diethyl ester or malonic acid dibutyl ester. In certain preferred embodiments of the present invention, the blocking agent is selected from ϵ-caprolactam, 1,2,4-triazole, methyl ethyl ketoxime, diisopropylamine, 3,5-dimethylpyrazole, and diethyl malonate.

Suitable monoisocyanate compounds include, but are not limited to those obtained by reacting aliphatic or aromatic monoamines with phosgene and those obtained by reacting one of the isocyanate groups of diisocyanate compounds with a hydroxy-containing compound; etc. U.S. Pat. No. 6,664,415 discloses a phosgene-free process for preparing organic monoisocyanates by thermally decomposing compounds containing biuret/urea groups and prepared in situ by the reaction of isocyanate compounds and primary monoamines

Suitable functional chemicals for inclusion in the isocyanate-attached functional chemical include, but are not limited to, demulsifiers, emulsifiers, corrosion inhibitors, dispersants, filtrate reducers, water clarifiers/deoilers, fluid-loss additives, flocculants, foaming agents, defoaming agents, scale inhibitors, shale control inhibitors, viscosifiers, thinners, wax inhibitors, asphaltene inhibitors, hydrate inhibitors, biocides, rheology modifiers, trace/tagging agents, co-surfactants, co-solvents, H₂S scavengers, ion exchange resins, cement gas blocking agents, cement retarders, de-watering additives, bentonite extenders, friction reducers, gelling agents, gel breakers, iron control additives, clay stabilizers, and iron sulfide dissolvers. Those functional chemicals lacking an active hydrogen may be modified so that they do contain an active hydrogen by methods known to those skilled in the art.

In some embodiments, demulsifiers are used to separate emulsions and include but are not limited to, acid catalyzed phenol-formaldehyde resins, base catalyzed phenol-formaldehyde resins, epoxy resins, polyethyleneimines, polyamines, di-epoxides, polyols, and dendrimers soap, naphthenic acid salts and alkylaryl sulphonate, sulphated castor oil petroleum sulphonates, derivatives of sulpho-acid oxidized castor oil and sulphosuccinic acid ester, fatty acids, fatty alcohols, alkylphenols, ethylene oxide, propylene oxide copolymer, alkoxylated cyclic p-alkylphenol formaldehyde resins, amine alkoxylate, alkoxylated cyclic p-alkylphenol formaldehyde resins, polyesteramine and blends.

In various embodiments, emulsifiers have molecules which consist of both a hydrophilic and a lipophilic part, allowing them to be surface-active and include, but are not limited to, calcium fatty-acid soaps made from various fatty acids and lime, or derivatives such as amides, amines, amidoamines and imidazolines made by reactions of fatty acids and various ethanolamine compounds.

In certain embodiments, the isocyanate-attached functional chemical is a corrosion inhibitor. Corrosion inhibitors are film-forming surfactants that form water-impermeable barriers on metal surfaces and include, but are not limited to, corrosion inhibitors include, but are not limited to quaternary ammonium salts such as chloride, bromides, iodides, dimethylsulfates, diethylsulfates, nitrites, bicarbonates, carbonates, hydroxides, alkoxides, or the like, or mixtures or combinations thereof; salts of nitrogen bases; or mixtures or combinations thereof.

In various embodiments, dispersants may be the isocyanate-attached functional chemical. Dispersants include, but are not limited to, complexes of tetravalent zirconium and ligands selected from organic acids such as citric, tartaric, malic, and lactic and a complex of aluminum and citric acid. A mixture of sulfonated styrene-maleic anhydride copolymer and polymers prepared from acrylic acid or acrylamide and their derivatives.

In some embodiments, the isocyanate-attached functional chemicals are filtrate reducers. Suitable filtrate reducers include, but are not limited to, bentonite, various manufactured polymers, starches, and thinners or deflocculants.

In various embodiments, water clarifiers and/or deoilers may be the isocyanate-attached functional chemical. Water clarifiers/deoilers describe compounds which are applied to break emulsions and separate the oil phase from the water, thereby, making the water “clearer” such as cationic poly-amines

Some embodiments of the present invention include fluid-loss additives as the isocyanate-attached functional chemical. Fluid-loss additives include, but are not limited to, starches, calcium carbonate-starch mixtures, guar gum, gum acacia, alginates, biopolymers, polyglycolic acids, polylactic acids, and mixtures thereof.

In various embodiments, flocculants may be the isocyanate-attached functional chemical. Suitable flocculants include, but are not limited to, salt, hydrated lime, gypsum (hydrated calcium sulfate), and synthetic polymers. Guar gum and some acrylic polymers are also very effective flocculants when used in low concentrations.

In certain embodiments of the present invention, the isocyanate-attached functional chemical may be a foaming agent. Foaming agents include suitable sodium salts of α-olefin sulfonates (AOSs), including, without limitation, any α-olefin sulfonate. Preferred AOSs including short chain α-olefin sulfonates having between about 2 and about 10 carbon atoms, particularly, between 4 and 10 carbon atoms, longer chain α-olefin sulfonates having between about 10 and about 24 carbon atoms, particularly, between about 10 and 16 carbon atoms or mixtures or combinations thereof.

In other embodiments, the isocyanate-attached functional chemical is a defoaming agent. Suitable defoaming agents include, but are not limited to, insoluble oils, polydimethylsiloxanes and other silicones, certain alcohols, stearates and glycols.

Certain embodiments may have a scale inhibitor as the isocyanate-attached functional chemical. Scale inhibitors including chelating agents, may be Na, K or NH₄⁺ salts of EDTA (ethylenediaminetetraacetic acid); Na, K or NH₄⁺ salts of NTA (nitrilotriacetic acid); Na, K or NH₄⁺ salts of erythorbic acid; Na, K or NH₄⁺ salts of thioglycolic acid (TGA); Na, K or NH₄⁺ salts of hydroxy acetic acid; Na, K or NH₄⁺ salts of citric acid; Na, K or NH₄⁺ salts of tartaric acid or other similar salts or mixtures or combinations thereof. Phosphates, e.g., sodium hexamethylphosphate, linear phosphate salts, salts of polyphosphoric acid, phosphonates, e.g., nonionic such as HEDP (hydroxyethylidene diphosphoric acid), PBTC (phosphoisobutane, tricarboxylic acid), amino phosphonates of: MEA (monoethanolamine), NH₃, EDA (ethylene diamine), bishydroxyethylene diamine, bisaminoethylether, DETA (diethylenetriamine), HMDA (hexamethylene diamine), hyper homologues and isomers of HMDA, polyamines of EDA and DETA, diglycolamine and homologues, or similar polyamines or mixtures or combinations thereof; phosphate esters, e.g., polyphosphoric acid esters or phosphorus pentoxide (P₂O₅) esters of: alkanol amines such as MEA, DEA, triethanol amine (TEA), bishydroxyethylethylene diamine; ethoxylated alcohols, glycerin, glycols such as EG (ethylene glycol), propylene glycol, butylene glycol, hexylene glycol, trimethylol propane, pentaerythritol, neopentyl glycol or the like; tris & tetrahydroxy amines; ethoxylated alkyl phenols (limited use due to toxicity problems), ethoxylated amines such as monoamines such as MDEA and higher amines from 2 to 24 carbons atoms, diamines 2 to 24 carbons carbon atoms, or the like; polymers, e.g., homopolymers of aspartic acid, soluble homopolymers of acrylic acid, copolymers of acrylic acid and methacrylic acid, terpolymers of acylates, AMPS, etc., hydrolyzed polyacrylamides, poly malic anhydride (PMA); or the like; or mixtures or combinations thereof.

In various embodiments, the isocyanate-attached functional chemical be a shale control inhibitor. Suitable shale control inhibitors include, but are not limited to, formates and anionic polymers/co-polymers.

In certain embodiments, the isocyanate-attached functional chemical is a thinner. Suitable thinners include, but are not limited to, plant tannins, phosphates, modified lignosulfonates; and low-molecular-weight synthetic water-soluble polymers.

In various embodiments, the isocyanate-attached functional chemical may be a wax inhibitor. Suitable wax inhibitors include, but are not limited to, styrene ester copolymers and terpolymers, esters, novalacs, polyalkylated phenol, and fumerate-vinyl acetate copolymers.

Suitable asphaltene inhibitors as the isocyanate-attached functional chemical include, but are not limited to, alkylphenol/aldehyde resins and sulfonated variants of these resins; polyolefin esters, amides, or imides with alkyl, alkylene phenyl, or alkylene pyridyl functional groups; alkenyl/vinylpyrolidone copolymers; graft polymers of polyolefins with maleic anhydride or vinylimidazole; hyperbranched polyesterimides; lignosulfonates; and polyalkoxylated asphaltenes.

Various embodiments of the present invention have hydrate inhibitors as the isocyanate-attached functional chemical. Suitable hydrate inhibitors include, but are not limited to, alkylated ammonium compounds, alkylated phosphonium compounds, alkylated sulfonium compounds, or any combination thereof. In some embodiments, the coating comprises tetrabutylammonium bromide. Water soluble polymers or copolymers of acrylamide, n-vinylamide maleimide, vinyllactam maleamide, alkenyl cyclic imino ether maleimide or other such polymers can also be used. Examples of hydrate anti-agglomerates include, but are not limited to, dodecyl-2-(2-caprolactamyl) ethanamide.

In certain embodiments of the present invention, the isocyanate-attached functional chemical is a biocide. Biocides are often used at low concentrations in the hydraulic fracturing fluid mixtures, at a level of about 0.001% in the fracturing fluid, which corresponds to approximately 0.01% of total proppant weight. Microorganisms can have a significant economic impact on the health and productivity of a well. For example, unchecked bacteria growth can result in “souring” of wells, where the bacteria produces hydrogen sulfide as a waste product of their metabolic function. Such sour gases in the produced fluids are highly undesirable and can be a source for corrosion in the production equipment as well as a cost for sulfur removal from the produced hydrocarbons.

Biocides can be divided into two types, non-oxidating and oxidating. Non-oxidating biocides include, but are not limited to, aldehydes, quaternary phosphonium compounds, quaternary ammonium surfactants, cationic polymers, organic bromides, metronidazole, isothiazolones, isothiazolinones, thiones, organic thiocyanates, phenolics, alkylamines, diamines, triamines, dithiocarbamates, 2-(decylthio)ethanamine (DTEA) and its hydrochloride, and triazine derivatives.

Oxidating biocides include, but are not limited to, hypochlorite and hypobromite salts, stabilized bromine chloride, hydroxyl radicals, chloramines, chloroisocyanurates, and halogen-containing hydantoins.

Exemplary biocidal agents include, but are not limited to: 2,2-dibromo-3-nitrilopropionamide (CAS 10222-01-2); glutaraldehyde (CAS 111-30-8); 2-bromo-2-cyanoacetamide (CAS 1113-55-9); caprylic alcohol (CAS 111-87-5); triethylene glycol (CAS 112-27-6); sodium dodecyl diphenyl ether disulfonate (CAS 119345-04-9); 2-amino-2-methyl-1-propanol (CAS 124-68-5); ethylenediaminetetraacetate (CAS 150-38-9); 5-chloro-2-methyl-4-isothiazolin-3-one (CAS 26172-55-4); benzisothiazolinone and other isothiazolinones (CAS 2634-33-5); ethoxylated oleylamine (CAS 26635-93-8); 2-methyl-4-isothiazolin-3-one (CAS 2682-20-4); formaldehyde (CAS 30846-35-6); dibromoacetonitrile (CAS 3252-43-5); dimethyl oxazolidine (CAS 51200-87-4); 2-bromo-2-nitro-1,3-propanediol (CAS 52-51-7); tetrahydro-3,5-dimethyl-2h-1,3,5-thia (CAS 533-73-2); 3,5-dimethyltetrahydro-1,3,5-thiadiazine-2-thione (CAS 533-74-4); tetrakis hydroxymethyl-phosphonium sulfate (CAS 55566-30-8); formaldehyde amine (CAS 56652-26-7); quaternary ammonium chloride (CAS 61789-71-1); C₆-C₁₂ ethoxylated alcohols (CAS 68002-97-1); benzalkonium chloride (CAS 68424-85-1); C₁₂-C₁₄ ethoxylated alcohols (CAS 68439-50-9); C₁₂-C₁₆ ethoxylated alcohols (CAS 68551-12-2); oxydiethylene bis(alkyldimethyl ammonium chloride) (CAS 68607-28-3); didecyl dimethyl ammonium chloride (CAS 7173-51-5); 3,4,4-trimethyl oxazolidine (CAS 75673-43-7); cetylethylmorpholinium ethyl sulfate (CAS 78-21-7); and tributyltetradecylphosphonium chloride (CAS 81741-28-8), or any combination thereof.

In various embodiments of the invention, the isocyanate-attached functional chemical is a rheology modifier. Synthetic rheology modifier polymers fall within one of four categories: alkali-soluble emulsion (“ASE”) polymers, hydrophobically-modified alkali-soluble emulsion (“HASE”) polymers, hydrophobically-modified ethoxylated urethane (“HEUR”) polymers, and hydrophobically-modified nonionic polyol (“HNP”) polymers. HASE are typically low viscosity emulsions at low pH levels. To increase viscosity, the emulsion is neutralized to pH above 7, preferably above 8. When it is neutralized, HASE swells and viscosifies. The hydrophobic modifications, for example, the terpene alkoxylate components acting like stickers and the acrylate-based extended hydrophilic brushes are responsible for creating the swollen three dimensional structure as viscous gels. HASE and ASE polymers, such as those described in, U.S. Pat. Nos. 3,035,004; 5,292,843; 6,897,253; 7,288,616; 7,378,479, and US Published Patent Application No. 2006/0270563, are suitable rheology modifiers in the present invention.

Trace/tagging agents may be the isocyanate-attached functional chemical in some embodiments of the invention and include, but are not limited to, dyes, fluorescer or other chemicals which can be detected using spectroscopic analytical methods such as UV-visible, fluorescence or phosphorescence. Compounds of lanthanide elements may be used as tracers because they have distinctive spectra. A tracer may be a chemical with distinctive features which enables it to be distinguished by another analytical technique such as GC-MS. Such chemicals include fluorocarbons and fluoro-substituted aromatic acids. Radio-isotopes may be used as tracers. Salts of ions which do not occur naturally in subterranean reservoirs, such as iodides and thiocyanates may also be used as a tracer.

Various embodiments of the invention have co-surfactants as the isocyanate-attached functional chemical. Co-surfactants include, but are not limited to, sodium dodecylbenzene sulfonate (SDBS), those having a SDBS-like structure having a saturated or unsaturated, branched or straight-chained C₆ to C₁₆ chain; further examples of this type of co-surfactant are those having a saturated or unsaturated, branched or straight-chained C₈ to C₁₆ chain. Other suitable examples of this type of co-surfactant, are certain chelating agents such as trisodium hydroxyethylethylenediamine triacetate.

Some embodiments of the present invention include co-solvents as the isocyanate-attached functional chemical. Co-solvents include, but are not limited to, short chain alkyl alcohols and glycols and combinations thereof. In some embodiments, the co-solvents comprise methanol, ethanol, isopropanol, 1,2-pentanediol, propylene glycol, and triethylene glycol and/or combinations thereof.

Hydrogen sulfide is a toxic chemical that is also corrosive to metals. The presence of hydrogen sulfide in hydrocarbon reservoirs raises the cost of production, transportation and refining due to increased safety and corrosion prevention requirements. Depending on the concentration of hydrogen sulfide in the fractured reservoir, the concentrations of the scavengers in the proppant coating can be varied to remove more or less hydrogen sulfide. In sufficient volume, proppants with sulfide scavenging capabilities can reduce the concentration from levels that pose safety hazards (in the range of 500-1000 ppm) to levels where the sulfides are only a nuisance (1-20 ppm). If the surface area of the proppants is high and dispersion of the scavengers is good, high efficiencies in hydrogen sulfide reaction and removal are possible. Suitable H₂S scavengers include, but are not limited to, quinones, benzoquinone, napthoquinones, agents containing quinone functional groups, such as chloranil, 2,3-dichloro-5,6-dicyanobenzoquinone, anthroquinone, and the like, or any combination thereof.

In certain embodiments of the present invention, the isocyanate-attached functional chemical is an ion exchange resin. Synthetic ion exchange resins are often a crosslinked polymer network to which are attached ionized or ionizable groups. In the case of cation exchange resins, these groups are acidic groups (e.g., —SO₃H, —PO₃H₂, —CO₂M, and phenolic hydroxyl) whereas in anion exchange resins the groups are basic in character (e.g., quaternary ammonium, aliphatic or aromatic amine groups). In the synthesis of ion exchange resins, the ionizable and contaminant removal functional groups may be attached to the monomers or intermediates used in preparation of the crosslinked polymer, or they may be introduced subsequently into a preformed polymer. As those skilled in the art are aware such are examples only and other anionic and cationic resins can be used.

The isocyanate-attached functional chemical is a cement gas blocking agent in certain embodiments of the invention. Gas blocking agents prevent gas channeling and include, but are not limited to BASOBLOCK styrene-butadiene lattices and PARAGAS which is a modified polyethylenimine, both from BASF, and GAS-CHEK from Halliburton.

Various embodiments of the invention have a cement retarder as the isocyanate-attached functional chemical. A cement retarder is a chemical used to increase the thickening time of a cement composition. Suitable cement retarders include, but are not limited to, saccharide compounds, polysaccharides organophosphates (such as alkylene phosphonic acids), inorganic compounds (such as acids and accompanying salts, sodium chloride, and oxides of zinc and lead).

In certain embodiments of the invention, a de-watering additive is the isocyanate-attached functional chemical. De-watering additives include, but are not limited to, polyacrylamide and polyacrylate polymers and copolymers.

Bentonite extenders are the isocyanate-attached functional chemicals in some embodiments and can be either salts or polymers which enhance viscosity buildup by slightly flocculating the bentonite suspension. More effective extenders than inorganic salts are the high molecular weight linear polymers.

Certain embodiments of the inventive isocyanate-attached functional chemical are friction reducers. Friction reducers include, but are not limited to, polyacrylamide and polyacrylate polymers and copolymers at low concentrations for all temperatures ranges.

Various embodiments of the invention include one or more gelling agents as the isocyanate-attached functional chemical. Gelling agents include, but are not limited to, guar gums, hydroxypropyl guar, carboxymethyl hydroxypropyl guar, carboxymethyl guar, and carboxymethyl hydroxyethyl cellulose. Suitable hydratable polymers may also include synthetic polymers, such as polyvinyl alcohol, polyacrylamides, poly-2-amino-2-methyl propane sulfonic acid, and various other synthetic polymers and copolymers. Other examples of such polymer include, without limitation, guar gums, high-molecular weight polysaccharides composed of mannose and galactose sugars, or guar derivatives such as hydropropyl guar (HPG), carboxymethyl guar (CMG), carboxymethylhydropropyl guar (CMHPG), hydroxyethylcellulose (HEC), hydroxypropylcellulose (HPC), carboxymethylhydroxyethylcellulose (CMHEC), xanthan, scleroglucan, polyacrylamide, polyacrylate polymers and copolymers.

A gel breaker is the isocyanate-attached functional chemical in some embodiments of the present invention. Gel breakers are either oxidants or enzymes which operate to degrade the polymeric gel structure. Most degradation or “breaking” is caused by oxidizing agents, such as persulfate salts (used either as is or encapsulated), chromous salts, organic peroxides or alkaline earth or zinc peroxide salts, or by enzymes.

In certain embodiments of the isocyanate-attached functional chemical of the present invention is an iron control agent. Suitable iron control agents for use in accordance with the present disclosure include but are not limited to those available from Halliburton Energy Services, Duncan, Okla., and include: FE-2 Iron sequestering agent, FE-2A Buffering agent, FE-3 Iron control agent, FE-3A Iron control agent, FE-5A Iron control agent, FERCHEK Ferric iron inhibitor, FERCHEK A reducing agent, and FERCHEK SC Iron control system. Other suitable iron control agents include those described in U.S. Pat. Nos. 6,315,045, 6,525,011, 6,534,448, and 6,706,668, the relevant disclosures of which are hereby incorporated by reference.

Other suitable iron control agents for use in accordance with the present invention include chelating agents, such as TRILON-B SP (available from BASF), an organic chelating agent, as well as other, similar chelating agents, including nitrilotri-acetate (NTA), tetrasodium ethylenediaminetetraacetate (EDTA), HEDTA, and DTPA, preferably EDTA (1-50 wt. %), as well as biodegradable chelating agents such as methyl glycine diacetic acid (MGDA, available as TRILON M), sodium glucoheptonate (Burco BSGH400), disodium hydroxymethyl-iminodiacetic acid (XUS 40855.01), imino disuccinic acid (BAYPURE CX 100/34 or BAYPURE CX 100 Solid G), EDDS ([S,S]-ethylenediamine-N,N′-disuccinic acid) (OCTAQUEST A65 or OCTAQUES t E30), citric acid, glycolic acid and lactic acid.

Other suitable iron control agents for use with the present invention include a number of organic acids, including ascorbic acid, erythorbic acid, and alkali metal salts thereof, complexing agents of the soluble forms of iron, such as the aminopolycarboxylic acid derivatives, citric acid, acetic acid or salicylic acid, and triethanolamine

In various embodiments, the isocyanate-attached functional chemical may comprise a clay stabilizer. Suitable clay stabilizers include, but are not limited to, flax seed gum and up to 10,000 ppm of potassium or ammonium cations, the use of an acid salt of alkaline esters, the use of aliphatic hydroxy acids with between 2-6 carbon atoms, cationic allyl ammonium halide salts, poly allyl ammonium halide salts, polyols containing at least 1 nitrogen atom preferably from a diamine, primary diamine salt with a chain length of 8 or less, quaternized trihydroxyalkylamines or choline derivatives, and quaternary amine-based cationic polyelectrolyte and salts. The cation of the salts may be a divalent salt cation, a choline cation, or certain N-substituted quaternary ammonium salt cations.

In some embodiments of the isocyanate-attached functional chemical may be an iron sulfide dissolver. An agent for treating such deposits is acrolein, but it also has health, safety and environmental problems. Tris (hydroxymethyl) phosphine has been found to be an effective chemical for removing iron sulfide deposits.

In addition to a dispersion, a coating composition may include other desired additives or auxiliaries. Suitable additives and auxiliaries include, but are not limited to, fillers, wetting agents, thickeners, surfactants, and colorants.

Although primarily exemplified herein in connection with polyurethane dispersions and blends containing polyurethane dispersions, the present invention is not intended to be so limited. The present invention also encompasses acrylate dispersions and styrene butadiene rubber (“SBR”) latex dispersions as the resin-containing dispersion, either alone or in combination with one or more polyurethane dispersions.

Further, in various embodiments, the isocyanate-attached functional chemical may be used without inclusion in a dispersion or the isocyanate-attached functional chemical may be introduced as a dry powder or a liquid into the downhole and allowed to mix with the fluids in the well such as fracing fluid, water, hydrocarbons, etc.

In those embodiments of the present invention utilizing proppants coated with the isocyanate-attached functional chemical of the present invention, the proppants may be introduced into the downhole formation via a pump. The pump may be a high pressure pump in some embodiments. As used herein, the term “high pressure pump” means a pump that is capable of delivering a fluid downhole at a pressure of about 1000 psi (6.89 MPa) or greater. A high pressure pump may be used to introduce the fluids to a subterranean formation at or above a fracture gradient of the subterranean formation, but it may also be used in cases where fracturing is not desired. In some embodiments, the high pressure pump may be capable of fluidly conveying particulate matter, such as the proppant described in the embodiments herein, into the subterranean formation. Suitable high pressure pumps will be known to those skilled in the art and may include, but are not limited to, floating piston pumps and positive displacement pumps.

In certain embodiments, the pump may be a low pressure pump. As used herein, the term “low pressure pump” means a pump that operates at a pressure of about 1000 psi (6.89 MPa) or less. In some embodiments, the low pressure pump may be configured to convey the fluids to the high pressure pump. In such embodiments, the low pressure pump may “step up” the pressure of the treatment fluids before reaching the high pressure pump.

In certain embodiments, the systems described herein can further comprise a mixing tank that is upstream of the pump and in which the fluids are formulated. In various embodiments, the pump (e.g., a low pressure pump, a high pressure pump, or a combination thereof) may convey the fluids from the mixing tank or other source of the fluids to the well. In other embodiments, however, the fluids may be formulated offsite and transported to a worksite, in which case the fluid may be introduced to the well via the pump directly from its shipping container (e.g., a truck, a railcar, a barge, or the like) or from a transport pipeline. In either case, the fluids may be drawn into the pump, elevated to an appropriate pressure, and then introduced for delivery downhole.

### EXAMPLES

The non-limiting and non-exhaustive examples that follow are intended to further describe various non-limiting and non-exhaustive embodiments without restricting the scope of the embodiments described in this specification. All quantities given in “parts” and “percents” are understood to be by weight, unless otherwise indicated.

The present inventors envision that any functional chemical having an active hydrogen atom may be attached at that active hydrogen atom to a monoisocyanate or polyisocyanate as desired. Such functional chemicals include, but are not limited to, biocides, demulsifiers, emulsifiers, corrosion inhibitors, dispersants, filtrate reducers, water clarifiers/deoilers, fluid-loss additives, flocculants, foaming agents, defoaming agents, scale inhibitors, shale control inhibitors, viscosifiers, thinners, wax inhibitors, asphaltene inhibitors, hydrate inhibitors, biocides, rheology modifiers, trace/tagging agents, co-surfactants, co-solvents, H₂S scavengers, ion exchange resins, cement gas blocking agents, cement retarders, de-watering additives, bentonite extenders, friction reducers, gelling agents, gel breakers, iron control additives, clay stabilizers, and iron sulfide dissolvers.

Blocking isocyanates with compounds containing active hydrogens is well known in the art and is a common technique in the preparation of urethane based coatings.

Isocyanate intermediates are blocked, as shown in the schematic below, for room temperature stability yet are reactive at high temperatures.

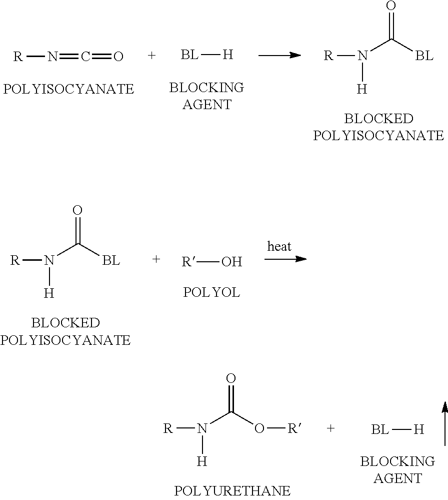

Below are some examples of blocking agents suitable for use in producing the isocyanate-attached functional chemicals of the present invention.

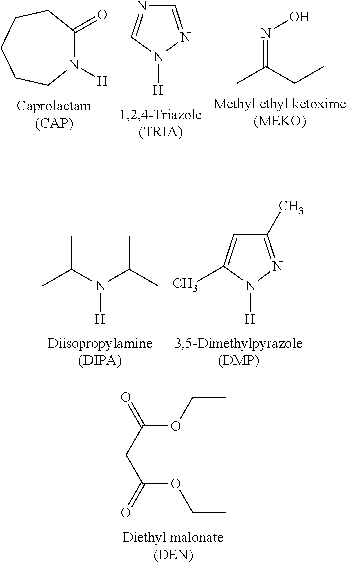

In addition to being a blocking agent, triazole is also a corrosion inhibitor for mild steel. Corrosion inhibitors may be use throughout all stages of the oil and gas well lifecycle: drilling; completion; cementing; stimulation and production.

This specification has been written with reference to various non-limiting and non-exhaustive embodiments. However, it will be recognized by persons having ordinary skill in the art that various substitutions, modifications, or combinations of any of the disclosed embodiments (or portions thereof) may be made within the scope of this specification. Thus, it is contemplated and understood that this specification supports additional embodiments not expressly set forth herein. Such embodiments may be obtained, for example, by combining, modifying, or reorganizing any of the disclosed steps, components, elements, features, aspects, characteristics, limitations, and the like, of the various non-limiting embodiments described in this specification. In this manner, Applicant(s) reserve the right to amend the claims during prosecution to add features as variously described in this specification, and such amendments comply with the requirements of 35 U.S.C. § 112(a), and 35 U.S.C. § 132(a).

Various aspects of the subject matter described herein are set out in the following numbered clauses:

1. A composition comprising an isocyanate, and a functional chemical, wherein the functional chemical is attached to the isocyanate at an active hydrogen atom of the functional chemical.

2. The composition according to clause 1, wherein the functional chemical is selected from the group consisting of demulsifiers, emulsifiers, corrosion inhibitors, dispersants, filtrate reducers, water clarifiers/deoilers, fluid-loss additives, flocculants, foaming agents, defoaming agents, scale inhibitors, shale control inhibitors, viscosifiers, thinners, wax inhibitors, asphaltene inhibitors, hydrate inhibitors, biocides, rheology modifiers, trace/tagging agents, co-surfactants, co-solvents, H₂S scavengers, ion exchange resins, cement gas blocking agents, cement retarders, de-watering additives, bentonite extenders, friction reducers, gelling agents, gel breakers, iron control additives, clay stabilizers, and iron sulfide dissolvers.

3. The composition according to clause 1, wherein the functional chemical is selected from the group consisting of aldehydes, quaternary phosphonium compounds, quaternary ammonium surfactants, cationic polymers, organic bromides, metronidazole, isothiazolones, isothiazolinones, thiones, organic thiocyanates, phenolics, alkylamines, diamines, triamines, dithiocarbamates, 2-(decylthio)ethanamine (DTEA) and its hydrochloride, triazine derivatives, hypochlorite and hypobromite salts, stabilized bromine chloride, hydroxyl radicals, chloramines, chloroisocyanurates, and halogen-containing hydantoins.

4. The composition according to clause 1, wherein the functional chemical is selected from the group consisting of 2,2-dibromo-3-nitrilopropionamide, glutaraldehyde, 2-bromo-2-cyanoacetamide, caprylic alcohol, triethylene glycol, sodium dodecyl diphenyl ether disulfonate, 2-amino-2-methyl-1-propanol, ethylenediaminetetraacetate, 5-chloro-2-methyl-4-isothiazolin-3-one, benzisothiazolinone, ethoxylated oleylamine, 2-methyl-4-isothiazolin-3-one, formaldehyde, dibromoacetonitrile, dimethyl oxazolidine, 2-bromo-2-nitro-1,3-propanediol, tetrahydro-3,5-dimethyl-2h-1,3,5-thia, 3,5-dimethltetrahydro-1,3,5-thiadiazine-2-thione, tetrakis hydroxymethyl-phosphonium sulfate, formaldehyde amine, quaternary ammonium chloride, C₆-C₁₂ ethoxylated alcohols, benzalkonium chloride, C₁₂-C₁₄ ethoxylated alcohols, C₁₂-C₁₆ ethoxylated alcohols, oxydiethylene bis(alkyldimethyl ammonium chloride), didecyl dimethyl ammonium chloride, 3,4,4-trimethyl oxazolidine, cetylethylmorpholinium ethyl sulfate, and tributyltetradecylphosphonium chloride or any combination thereof.

5. The composition according to one of clauses 1 to 4, wherein the isocyanate is a monoisocyanate.

6. The composition according to one of clauses 1 to 4, wherein the isocyanate is a polyisocyanate.

7. One of a coating, an adhesive, or a sealant including the composition according to one of clauses 1 to 6.

8. A process comprising attaching a functional chemical to an isocyanate at an active hydrogen atom of the functional chemical.

9. The process according to clause 8, wherein the functional chemical is selected from the group consisting of demulsifiers, emulsifiers, corrosion inhibitors, dispersants, filtrate reducers, water clarifiers/deoilers, fluid-loss additives, flocculants, foaming agents, defoaming agents, scale inhibitors, shale control inhibitors, viscosifiers, thinners, wax inhibitors, asphaltene inhibitors, hydrate inhibitors, biocides, rheology modifiers, trace/tagging agents, co-surfactants, co-solvents, H₂S scavengers, ion exchange resins, cement gas blocking agents, cement retarders, de-watering additives, bentonite extenders, friction reducers, gelling agents, gel breakers, iron control additives, clay stabilizers, and iron sulfide dissolvers.

10. The process according to clause 8, wherein the functional chemical is selected from the group consisting of aldehydes, quaternary phosphonium compounds, quaternary ammonium surfactants, cationic polymers, organic bromides, metronidazole, isothiazolones, isothiazolinones, thiones, organic thiocyanates, phenolics, alkylamines, diamines, triamines, dithiocarbamates, 2-(decylthio)ethanamine (DTEA) and its hydrochloride, triazine derivatives, hypochlorite and hypobromite salts, stabilized bromine chloride, hydroxyl radicals, chloramines, chloroisocyanurates, and halogen-containing hydantoins.

11. The process according to clause 8, wherein the functional chemical is selected from the group consisting of 2,2-dibromo-3-nitrilopropionamide, glutaraldehyde, 2-bromo-2-cyanoacetamide, caprylic alcohol, triethylene glycol, sodium dodecyl diphenyl ether disulfonate, 2-amino-2-methyl-1-propanol, ethylenediaminetetraacetate, 5-chloro-2-methyl-4-isothiazolin-3-one, benzisothiazolinone, ethoxylated oleylamine, 2-methyl-4-isothiazolin-3-one, formaldehyde, dibromoacetonitrile, dimethyl oxazolidine, 2-bromo-2-nitro-1,3-propanediol, tetrahydro-3,5-dimethyl-2h-1,3,5-thia, 3,5-dimethyltetrahydro-1,3,5-thiadiazine-2-thione, tetrakis hydroxymethyl-phosphonium sulfate, formaldehyde amine, quaternary ammonium chloride, C₆-C₁₂ ethoxylated alcohols, benzalkonium chloride, C₁₂-C₁₄ ethoxylated alcohols, C₁₂-C₁₆ ethoxylated alcohols, oxydiethylene bis(alkyldimethyl ammonium chloride), didecyl dimethyl ammonium chloride, 3,4,4-trimethyl oxazolidine, cetylethylmorpholinium ethyl sulfate, and tributyltetradecylphosphonium chloride or any combination thereof.

12. The process according to one of clauses 8 to 11, wherein the isocyanate is a monoisocyanate.

13. The process according to one of clauses 8 to 11, wherein the isocyanate is a polyisocyanate.

14. A hydraulic fracturing fluid containing a composition comprising an isocyanate, and a functional chemical, wherein the functional chemical is attached to the isocyanate at an active hydrogen atom of the functional chemical.

15. The hydraulic fracturing fluid according to clause 14, wherein the functional chemical is selected from the group consisting of demulsifiers, emulsifiers, corrosion inhibitors, dispersants, filtrate reducers, water clarifiers/deoilers, fluid-loss additives, flocculants, foaming agents, defoaming agents, scale inhibitors, shale control inhibitors, viscosifiers, thinners, wax inhibitors, asphaltene inhibitors, hydrate inhibitors, biocides, rheology modifiers, trace/tagging agents, co-surfactants, co-solvents, H₂S scavengers, ion exchange resins, cement gas blocking agents, cement retarders, de-watering additives, bentonite extenders, friction reducers, gelling agents, gel breakers, iron control additives, clay stabilizers, and iron sulfide dissolvers.

16. The hydraulic fracturing fluid according to clause 14, wherein the functional chemical is selected from the group consisting of aldehydes, quaternary phosphonium compounds, quaternary ammonium surfactants, cationic polymers, organic bromides, metronidazole, isothiazolones, isothiazolinones, thiones, organic thiocyanates, phenolics, alkylamines, diamines, triamines, dithiocarbamates, 2-(decylthio)ethanamine (DTEA) and its hydrochloride, triazine derivatives, hypochlorite and hypobromite salts, stabilized bromine chloride, hydroxyl radicals, chloramines, chloroisocyanurates, and halogen-containing hydantoins.

17. The hydraulic fracturing fluid according to clause 14, wherein the functional chemical is selected from the group consisting of 2,2-dibromo-3-nitrilopropionamide, glutaraldehyde, 2-bromo-2-cyanoacetamide, caprylic alcohol, triethylene glycol, sodium dodecyl diphenyl ether disulfonate, 2-amino-2-methyl-1-propanol, ethylenediaminetetraacetate, 5-chloro-2-methyl-4-isothiazolin-3-one, benzisothiazolinone, ethoxylated oleylamine, 2-methyl-4-isothiazolin-3-one, formaldehyde, dibromoacetonitrile, dimethyl oxazolidine, 2-bromo-2-nitro-1,3-propanediol, tetrahydro-3,5-dimethyl-2h-1,3,5-thia, 3,5-dimethyltetrahydro-1,3,5-thiadiazine-2-thione, tetrakis hydroxymethyl-phosphonium sulfate, formaldehyde amine, quaternary ammonium chloride, C₆-C₁₂ ethoxylated alcohols, benzalkonium chloride, C₁₂-C₁₄ ethoxylated alcohols, C₁₂-C₁₆ ethoxylated alcohols, oxydiethylene bis(alkyldimethyl ammonium chloride), didecyl dimethyl ammonium chloride, 3,4,4-trimethyl oxazolidine, cetylethylmorpholinium ethyl sulfate, and tributyltetradecylphosphonium chloride or any combination thereof.

18. The hydraulic fracturing fluid according to one of clauses 14 to 17, wherein the isocyanate is a monoisocyanate.

19. The hydraulic fracturing fluid according to one of clauses 14 to 17, wherein the isocyanate is a polyisocyanate.

20. An oil and/or gas well comprising a hydraulic fracturing fluid containing a composition comprising an isocyanate, and a functional chemical, wherein the functional chemical is attached to the isocyanate at an active hydrogen atom of the functional chemical.

21. The oil and/or gas well according to clause 20, wherein the functional chemical is selected from the group consisting of demulsifiers, emulsifiers, corrosion inhibitors, dispersants, filtrate reducers, water clarifiers/deoilers, fluid-loss additives, flocculants, foaming agents, defoaming agents, scale inhibitors, shale control inhibitors, viscosifiers, thinners, wax inhibitors, asphaltene inhibitors, hydrate inhibitors, biocides, rheology modifiers, trace/tagging agents, co-surfactants, co-solvents, H₂S scavengers, ion exchange resins, cement gas blocking agents, cement retarders, de-watering additives, bentonite extenders, friction reducers, gelling agents, gel breakers, iron control additives, clay stabilizers, and iron sulfide dissolvers.

22. The oil and/or gas well according to clause 20, wherein the functional chemical is selected from the group consisting of aldehydes, quaternary phosphonium compounds, quaternary ammonium surfactants, cationic polymers, organic bromides, metronidazole, isothiazolones, isothiazolinones, thiones, organic thiocyanates, phenolics, alkylamines, diamines, triamines, dithiocarbamates, 2-(decylthio)ethanamine (DTEA) and its hydrochloride, triazine derivatives, hypochlorite and hypobromite salts, stabilized bromine chloride, hydroxyl radicals, chloramines, chloroisocyanurates, and halogen-containing hydantoins.

23. The oil and/or gas well according to clause 20, wherein the functional chemical is selected from the group consisting of 2,2-dibromo-3-nitrilopropionamide, glutaraldehyde, 2-bromo-2-cyanoacetamide, caprylic alcohol, triethylene glycol, sodium dodecyl diphenyl ether disulfonate, 2-amino-2-methyl-1-propanol, ethylenediaminetetraacetate, 5-chloro-2-methyl-4-isothiazolin-3-one, benzisothiazolinone, ethoxylated oleylamine, 2-methyl-4-isothiazolin-3-one, formaldehyde, dibromoacetonitrile, dimethyl oxazolidine, 2-bromo-2-nitro-1,3-propanediol, tetrahydro-3,5-dimethyl-2h-1,3,5-thia, 3,5-dimethyltetrahydro-1,3,5-thiadiazine-2-thione, tetrakis hydroxymethyl-phosphonium sulfate, formaldehyde amine, quaternary ammonium chloride, C₆-C₁₂ ethoxylated alcohols, benzalkonium chloride, C₁₂-C₁₄ ethoxylated alcohols, C₁₂-C₁₆ ethoxylated alcohols, oxydiethylene bis(alkyldimethyl ammonium chloride), didecyl dimethyl ammonium chloride, 3,4,4-trimethyl oxazolidine, cetylethylmorpholinium ethyl sulfate, and tributyltetradecylphosphonium chloride or any combination thereof.

24. The hydraulic fracturing fluid according to one of clauses 20 to 23, wherein the isocyanate is a monoisocyanate.

25. The hydraulic fracturing fluid according to one of clauses 20 to 23, wherein the isocyanate is a polyisocyanate.

26. In a process of conducting one or more stages of an oil and gas well lifecycle, the improvement comprising a step of adding a composition comprising an isocyanate, and a functional chemical, wherein the functional chemical is attached to the isocyanate at an active hydrogen atom of the functional chemical.

27. The process according to clause 26, wherein the stage is selected from the group consisting of drilling, completion, cementing, stimulation and production.

What is claimed is:

1. A composition comprising isocyanate, and a functional chemical, wherein the functional chemical is attached to the isocyanate at an active hydrogen atom of the functional chemical.

2. The composition according to claim 1, wherein the functional chemical is selected from the group consisting of demulsifiers, emulsifiers, corrosion inhibitors, dispersants, filtrate reducers, water clarifiers/deoilers, fluid-loss additives, flocculants, foaming agents, defoaming agents, scale inhibitors, shale control inhibitors, viscosifiers, thinners, wax inhibitors, asphaltene inhibitors, hydrate inhibitors, biocides, rheology modifiers, trace/tagging agents, co-surfactants, co-solvents, H₂S scavengers, ion exchange resins, cement gas blocking agents, cement retarders, de-watering additives, bentonite extenders, friction reducers, gelling agents, gel breakers, iron control additives, clay stabilizers, and iron sulfide dissolvers.

3. The composition according to claim 1, wherein the functional chemical is selected from the group consisting of aldehydes, quaternary phosphonium compounds, quaternary ammonium surfactants, cationic polymers, organic bromides, metronidazole, isothiazolones, isothiazolinones, thiones, organic thiocyanates, phenolics, alkylamines, diamines, triamines, dithiocarbamates, 2-(decylthio)ethanamine (DTEA) and its hydrochloride, triazine derivatives, hypochlorite and hypobromite salts, stabilized bromine chloride, hydroxyl radicals, chloramines, chloroisocyanurates, and halogen-containing hydantoins.

4. The composition according to claim 1, wherein the functional chemical is selected from the group consisting of 2,2-dibromo-3-nitrilopropionamide, glutaraldehyde, 2-bromo-2-cyanoacetamide, caprylic alcohol, triethylene glycol, sodium dodecyl diphenyl ether disulfonate, 2-amino-2-methyl-1-propanol, ethylenediaminetetraacetate, 5-chloro-2-methyl-4-isothiazolin-3-one, benzisothiazolinone, ethoxylated oleylamine, 2-methyl-4-isothiazolin-3-one, formaldehyde, dibromoacetonitrile, dimethyl oxazolidine, 2-bromo-2-nitro-1,3-propanediol, tetrahydro-3,5-dimethyl-2h-1,3,5-thia, 3,5-dimethyltetrahydro-1,3,5-thiadiazine-2-thione, tetrakis hydroxymethyl-phosphonium sulfate, formaldehyde amine, quaternary ammonium chloride, C₆-C₁₂ ethoxylated alcohols, benzalkonium chloride, C₁₂-C₁₄ ethoxylated alcohols, C₁₂-C₁₆ ethoxylated alcohols, oxydiethylene bis(alkyldimethyl ammonium chloride), didecyl dimethyl ammonium chloride, 3,4,4-trimethyl oxazolidine, cetylethylmorpholinium ethyl sulfate, and tributyltetradecylphosphonium chloride or any combination thereof.

5. The composition according to claim 1, wherein the isocyanate is a monoisocyanate.

6. The composition according to claim 1, wherein the isocyanate is a polyisocyanate.

7. One of a coating, an adhesive, or a sealant including the composition according to claim 1.

8. A process comprising attaching a functional chemical to an isocyanate at an active hydrogen atom of the functional chemical.

9. The process according to claim 8, wherein the functional chemical is selected from the group consisting of demulsifiers, emulsifiers, corrosion inhibitors, dispersants, filtrate reducers, water clarifiers/deoilers, fluid-loss additives, flocculants, foaming agents, defoaming agents, scale inhibitors, shale control inhibitors, viscosifiers, thinners, wax inhibitors, asphaltene inhibitors, hydrate inhibitors, biocides, rheology modifiers, trace/tagging agents, co-surfactants, co-solvents, H₂S scavengers, ion exchange resins, cement gas blocking agents, cement retarders, de-watering additives, bentonite extenders, friction reducers, gelling agents, gel breakers, iron control additives, clay stabilizers, and iron sulfide dissolvers.

10. The process according to claim 8, wherein the functional chemical is selected from the group consisting of aldehydes, quaternary phosphonium compounds, quaternary ammonium surfactants, cationic polymers, organic bromides, metronidazole, isothiazolones, isothiazolinones, thiones, organic thiocyanates, phenolics, alkylamines, diamines, triamines, dithiocarbamates, 2-(decylthio)ethanamine (DTEA) and its hydrochloride, triazine derivatives, hypochlorite and hypobromite salts, stabilized bromine chloride, hydroxyl radicals, chloramines, chloroisocyanurates, and halogen-containing hydantoins.

11. The process according to claim 8, wherein the functional chemical is selected from the group consisting of 2,2-dibromo-3-nitrilopropionamide, glutaraldehyde, 2-bromo-2-cyanoacetamide, caprylic alcohol, triethylene glycol, sodium dodecyl diphenyl ether disulfonate, 2-amino-2-methyl-1-propanol, ethylenediaminetetraacetate, 5-chloro-2-methyl-4-isothiazolin-3-one, benzisothiazolinone, ethoxylated oleylamine, 2-methyl-4-isothiazolin-3-one, formaldehyde, dibromoacetonitrile, dimethyl oxazolidine, 2-bromo-2-nitro-1,3-propanediol, tetrahydro-3,5-dimethyl-2h-1,3,5-thia, 3,5-dimethyltetrahydro-1,3,5-thiadiazine-2-thione, tetrakis hydroxymethyl-phosphonium sulfate, formaldehyde amine, quaternary ammonium chloride, C₆-C₁₂ ethoxylated alcohols, benzalkonium chloride, C₁₂-C₁₄ ethoxylated alcohols, C₁₂-C₁₆ ethoxylated alcohols, oxydiethylene bis(alkyldimethyl ammonium chloride), didecyl dimethyl ammonium chloride, 3,4,4-trimethyl oxazolidine, cetylethylmorpholinium ethyl sulfate, and tributyltetradecylphosphonium chloride or any combination thereof.

12. The process according to claim 8, wherein the isocyanate is a monoisocyanate.

13. The process according to claim 8, wherein the isocyanate is a polyisocyanate.

14. A hydraulic fracturing fluid containing a composition comprising an isocyanate, and a functional chemical, wherein the functional chemical is attached to the isocyanate at an active hydrogen atom of the functional chemical.

15. The hydraulic fracturing fluid according to claim 14, wherein the functional chemical is selected from the group consisting of demulsifiers, emulsifiers, corrosion inhibitors, dispersants, filtrate reducers, water clarifiers/deoilers, fluid-loss additives, flocculants, foaming agents, defoaming agents, scale inhibitors, shale control inhibitors, viscosifiers, thinners, wax inhibitors, asphaltene inhibitors, hydrate inhibitors, biocides, rheology modifiers, trace/tagging agents, co-surfactants, co-solvents, H2S scavengers, ion exchange resins, cement gas blocking agents, cement retarders, de-watering additives, bentonite extenders, friction reducers, gelling agents, gel breakers, iron control additives, clay stabilizers, and iron sulfide dissolvers.

16. The hydraulic fracturing fluid according to claim 14, wherein the functional chemical is selected from the group consisting of aldehydes, quaternary phosphonium compounds, quaternary ammonium surfactants, cationic polymers, organic bromides, metronidazole, isothiazolones, isothiazolinones, thiones, organic thiocyanates, phenolics, alkylamines, diamines, triamines, dithiocarbamates, 2-(decylthio)ethanamine (DTEA) and its hydrochloride, triazine derivatives, hypochlorite and hypobromite salts, stabilized bromine chloride, hydroxyl radicals, chloramines, chloroisocyanurates, and halogen-containing hydantoins.

17. The hydraulic fracturing fluid according to claim 14, wherein the functional chemical is selected from the group consisting of 2,2-dibromo-3-nitrilopropionamide, glutaraldehyde, 2-bromo-2-cyanoacetamide, caprylic alcohol, triethylene glycol, sodium dodecyl diphenyl ether disulfonate, 2-amino-2-methyl-1-propanol, ethylenediaminetetraacetate, 5-chloro-2-methyl-4-isothiazolin-3-one, benzisothiazolinone, ethoxylated oleylamine, 2-methyl-4-isothiazolin-3-one, formaldehyde, dibromoacetonitrile, dimethyl oxazolidine, 2-bromo-2-nitro-1,3-propanediol, tetrahydro-3,5-dimethyl-2h-1,3,5-thia, 3,5-dimethyltetrahydro-1,3,5-thiadiazine-2-thione, tetrakis hydroxymethyl-phosphonium sulfate, formaldehyde amine, quaternary ammonium chloride, C₆-C₁₂ ethoxylated alcohols, benzalkonium chloride, C₁₂-C₁₄ ethoxylated alcohols, C₁₂-C₁₆ ethoxylated alcohols, oxydiethylene bis(alkyldimethyl ammonium chloride), didecyl dimethyl ammonium chloride, 3,4,4-trimethyl oxazolidine, cetylethylmorpholinium ethyl sulfate, and tributyltetradecylphosphonium chloride or any combination thereof.

18. The hydraulic fracturing fluid according to claim 14, wherein the isocyanate is a monoisocyanate.

19. The hydraulic fracturing fluid according to claim 14, wherein the isocyanate is a polyisocyanate.

20. An oil and/or gas well comprising a hydraulic fracturing fluid containing a composition comprising an isocyanate, and a functional chemical, wherein the functional chemical is attached to the isocyanate at an active hydrogen atom of the functional chemical.

21. The oil and/or gas well according to claim 20, wherein the functional chemical is selected from the group consisting of demulsifiers, emulsifiers, corrosion inhibitors, dispersants, filtrate reducers, water clarifiers/deoilers, fluid-loss additives, flocculants, foaming agents, defoaming agents, scale inhibitors, shale control inhibitors, viscosifiers, thinners, wax inhibitors, asphaltene inhibitors, hydrate inhibitors, biocides, rheology modifiers, trace/tagging agents, co-surfactants, co-solvents, H₂S scavengers, ion exchange resins, cement gas blocking agents, cement retarders, de-watering additives, bentonite extenders, friction reducers, gelling agents, gel breakers, iron control additives, clay stabilizers, and iron sulfide dissolvers.

22. The oil and/or gas well according to claim 20, wherein the functional chemical is selected from the group consisting of aldehydes, quaternary phosphonium compounds, quaternary ammonium surfactants, cationic polymers, organic bromides, metronidazole, isothiazolones, isothiazolinones, thiones, organic thiocyanates, phenolics, alkylamines, diamines, triamines, dithiocarbamates, 2-(decylthio)ethanamine (DTEA) and its hydrochloride, triazine derivatives, hypochlorite and hypobromite salts, stabilized bromine chloride, hydroxyl radicals, chloramines, chloroisocyanurates, and halogen-containing hydantoins.

23. The oil and/or gas well according to claim 20, wherein the functional chemical is selected from the group consisting of 2,2-dibromo-3-nitrilopropionamide, glutaraldehyde, 2-bromo-2-cyanoacetamide, caprylic alcohol, triethylene glycol, sodium dodecyl diphenyl ether disulfonate, 2-amino-2-methyl-1-propanol, ethylenediaminetetraacetate, 5-chloro-2-methyl-4-isothiazolin-3-one, benzisothiazolinone, ethoxylated oleylamine, 2-methyl-4-isothiazolin-3-one, formaldehyde, dibromoacetonitrile, dimethyl oxazolidine, 2-bromo-2-nitro-1,3-propanediol, tetrahydro-3,5-dimethyl-2h-1,3,5-thia, 3,5-dimethyltetrahydro-1,3,5-thiadiazine-2-thione, tetrakis hydroxymethyl-phosphonium sulfate, formaldehyde amine, quaternary ammonium chloride, C₆-C₁₂ ethoxylated alcohols, benzalkonium chloride, C₁₂-C₁₄ ethoxylated alcohols, C₁₂-C₁₆ ethoxylated alcohols, oxydiethylene bis(alkyldimethyl ammonium chloride), didecyl dimethyl ammonium chloride, 3,4,4-trimethyl oxazolidine, cetylethylmorpholinium ethyl sulfate, and tributyltetradecylphosphonium chloride or any combination thereof.

24. The oil and/or gas well according to claim 20, wherein the isocyanate is a monoisocyanate.

25. The oil and/or gas well according to claim 20, wherein the isocyanate is a polyisocyanate.

26. In a process of conducting one or more stages of an oil and gas well lifecycle, the improvement comprising a step of adding a composition comprising an isocyanate, and a functional chemical, wherein the functional chemical is attached to the isocyanate at an active hydrogen atom of the functional chemical.

27. The process according to claim 26, wherein the stage is selected from the group consisting of drilling, completion, cementing, stimulation and production.
